(19) United States
(12) Patent Application Publication
(10) Pub. No.: US 2024/0133888 A1
(43) Pub. Date: Apr. 25, 2024

(54) NANOSTRUCTURE FOR DETECTING VIRUSES CONTAINING AMPHIPATHIC POLYMER AND DIAGNOSTIC PLATFORM USING THE SAME

(71) Applicant: KNU-INDUSTRY COOPERATION FOUNDATION, Chuncheon-si (KR)

(72) Inventors: HYUN OUK KIM, Chuncheon-si (KR); JAE WON CHOI, Chuncheon-si (KR); SO JIN SHIN, Chuncheon-si (KR); YU RIM AHN, Chuncheon-si (KR); HEE WON AN, Chuncheon-si (KR); MIN SE KIM, Chuncheon-si (KR); HAK SEON KIM, Chuncheon-si (KR)

(73) Assignee: KNU-INDUSTRY COOPERATION FOUNDATION, Chuncheon-si (KR)

(21) Appl. No.: 18/381,221

(22) Filed: Oct. 17, 2023

(30) Foreign Application Priority Data
Oct. 18, 2022 (KR) 10-2022-0135017
Dec. 26, 2022 (KR) 10-2022-0184549

(51) Int. Cl. G01N 33/569; G01N 33/543; G01N 33/549; G01N 33/552

(52) U.S. Cl. CPC G01N 33/56983; G01N 33/54346; G01N 33/549; G01N 33/552; B82Y 5/00

(57) ABSTRACT

The present disclosure relates to a nanostructure for detecting viruses including an amphipathic polymer, and a diagnostic platform using the same, wherein the nanostructure is capable of specifically detecting viruses through silica-based nanoparticles with excellent stability and high dispersion and a biocompatible amphipathic polymer, such that it is possible to develop a diagnostic platform with high sensitivity through binding and agglomeration of the nanostructure and viruses and enable rapid and accurate diagnosis of a target virus.

### FIELD OF THE INVENTION

The present disclosure relates to a nanostructure for detecting viruses containing an amphipathic polymer and a diagnostic platform using the same.

### DESCRIPTION OF THE RELATED ART

Diseases caused by viral infection, including coronavirus disease-19 (COVID-19), have been increasing in recent years. Of diseases caused by viruses, influenza, commonly known as “flu”, is an acute respiratory disease caused by influenza virus. Influenza is a highly contagious disease that causes small and large epidemics worldwide, infecting 10 to 20% of the population generally within 2 to 3 weeks once the outbreak begins.

When infected with the influenza virus, healthy people may experience symptoms such as chills, fever, headache, and cough for several days before recovering, but people with chronic lung disease, heart disease, and weakened immunity may develop complications such as pneumonia and die. Every year, 5 to 10% of the adult population and 20 to 30% of the pediatric population are infected with the influenza virus, reaching a maximum of 1 billion people, of which 150,000500,000 people die. Therefore, various technologies capable of detecting viruses are being developed.

As for existing onsite diagnosis, lateral flow immunoassay (LFIA) has been used as a major method that is widely used owing to advantages such as convenience and speed. However, in the case of existing rapid diagnostic kits, it has poor sensitivity as well as high rate of false positives and false negatives.

Therefore, it is necessary to develop nanoplatforms with high sensitivity and technologies that may quickly and accurately diagnose target viruses.

### PRIOR ART DOCUMENT

### Patent Document

Korean Patent No. 10-1546534 (Published on Jul. 2, 2015)

### SUMMARY

### Problem to be Solved by the Invention

In an attempt to quickly diagnose on site, an object of the present disclosure is to provide a nanostructure for detecting viruses containing an amphipathic polymer having an antibody specific to the viruses, and a diagnostic platform using the same.

### Means for Solving the Problem

In order to achieve the above object, the present disclosure provides a nanostructure for detecting viruses, including porous silica nanoparticles; an amphipathic polymer bound to a surface of the silica nanoparticles; and a virus antibody bound to the amphipathic polymer.

In addition, the present disclosure provides a composition for diagnosing viral infection including the above-described nanostructure for detecting viruses as an active ingredient.

In addition, the present disclosure provides a method of preparing a nanostructure for detecting viruses, including preparing porous silica nanoparticles; mixing the prepared silica nanoparticles with an amphipathic polymer to bind the amphipathic polymer; and mixing the amphipathic polymer-bound silica nanoparticles and a virus antibody to bind the antibody.

### Effects of the Invention

A nanostructure according to the present disclosure is capable of specifically detecting viruses through silica-based nanoparticles with excellent stability and high dispersion and a biocompatible amphipathic polymer, such that it is possible to develop a diagnostic platform with high sensitivity through binding and agglomeration of the nanostructure and viruses and enable rapid and accurate diagnosis of a target virus.

### BRIEF DESCRIPTION OF THE DRAWINGS

FIG. 1 shows a schematic diagram of a nanostructure for detecting viruses according to the present disclosure.

FIG. 2 shows results of dynamic light scattering (DLS) analysis and transmission electron microscope (TransTEM) imaging of silica nanoparticles and mesoporous silica nanoparticles according to the present disclosure.

FIG. 3 shows results of dynamic light scattering (DLS) analysis and transmission to electron microscope (TransTEM) imaging of PEGylated silica nanoparticles and PEGylated mesoporous silica nanoparticles according to the present disclosure.

FIG. 4 shows results of dynamic light scattering (DLS) analysis and transmission electron microscope (TransTEM) imaging of antibody-PEGylated silica nanoparticles and antibody-PEGylated mesoporous silica nanoparticles according to the present disclosure.

FIG. 5 shows results of dynamic light scattering (DLS) analysis of antibody-PEGylated silica nanoparticles and antibody-PEGylated mesoporous silica nanoparticles according to the present disclosure after binding and agglomerating with H1N1 virus.

FIG. 6 shows results of dynamic light scattering (DLS) analysis of antibody-PEGylated silica nanoparticles according to the present disclosure after binding and agglomerating with various concentrations of H1N1 virus.

FIG. 7 shows results of dynamic light scattering (DLS) analysis of antibody-PEGylated mesoporous silica nanoparticles according to the present disclosure after binding and agglomerating with various concentrations of H1N1 virus.

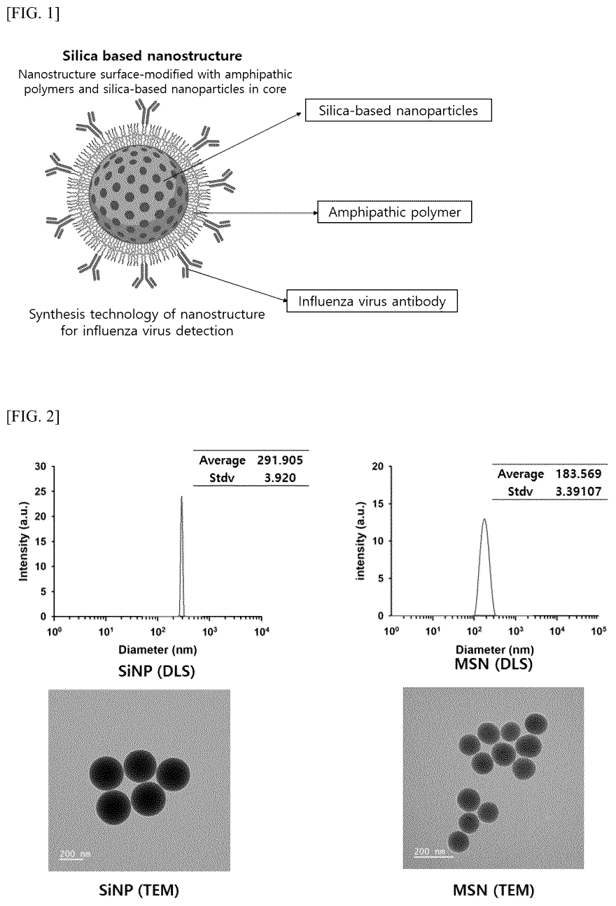

### DETAILED DESCRIPTION

Hereinafter, the present disclosure will be described in detail.

The present disclosure provides a nanostructure for detecting viruses, including porous silica nanoparticles; an amphipathic polymer bound to a surface of the silica nanoparticles; and a virus antibody bound to the amphipathic polymer.

The porous silica nanoparticles may have a diameter of 50 to 300 nm or 100 to 200 nm.

The porous silica nanoparticles ensure easy surface modification, excellent stability, and high dispersion.

The amphipathic polymer may include any one or more selected from the group consisting of silane-poly(ethylene glycol)-COOH, polyethyleneimine (PEI), chitosan, polypropylene imine, polyly sine, polyamidoamine, poly(allylamine), poly(di allyldimethyl ammonium chloride), poly(N-isopropyl acrylamide-co-acrylamide), poly(N-isopropylacrylamide-co-acrylic acid), diethylaminoethyl-dextran, poly(N-ethyl-vinylpyridinium bromide), poly(dimethylamino)ethyl methacrylate, poly(ethylene glycol)-co-poly(trimethylamino ethyl methacrylate chloride), and methoxy poly(ethylene glycol)-b-poly(D, L-lactide).

The amphipathic polymer is a biofriendly, surface-reactive derivative, which may specifically detect viruses by inhibiting nonspecific binding with other molecules.

The virus antibody may include antibodies of any one or more selected from the group consisting of influenza A virus, influenza B virus, dengue virus, human respiratory syncytial virus, norovirus, MERS coronavirus, SARS coronavirus, and SARS coronavirus-2.

The influenza A virus antibody may include any one or more selected from the group consisting of H1N1 virus antibody, H2N2 virus antibody, H3N2 virus antibody, H5N1 virus antibody, H7N9 virus antibody, H7N7 virus antibody, H1N2 virus antibody, H9N2 virus antibody, H7N2 virus antibody, H7N3 virus antibody, H5N2 virus antibody, and H10N7 virus antibody.

It is possible to detect viruses by specifically binding to the virus through the virus antibody, without requiring a lysis process by binding to the virus surface.

The nanostructure for detecting viruses of the present disclosure may be in a form in which a carboxyl group of the amphipathic polymer and the antibody are bound and linked.

The nanostructure for detecting viruses of the present disclosure may quickly and accurately detect viruses by binding and agglomerating specifically to viruses.

In addition, the present disclosure provides a composition for diagnosing viral infection, including the above-described nanostructure for detecting viruses as an active ingredient.

The corresponding features may be substituted in the above-described part.

In addition, the present disclosure provides a method of preparing a nanostructure for detecting virus, including: preparing porous silica nanoparticles; mixing the prepared silica nanoparticles with an amphipathic polymer to bind the amphipathic polymer; and mixing the amphipathic polymer-bound silica nanoparticles and an influenza A virus antibody to bind the antibody.

The preparing of the porous silica nanoparticles may be performed by adding a silica precursor to a mixture of surfactants, solvents, and catalysts to carry out a reaction and then washing the reactants.

Specifically, the surfactant may include one or more selected from the group consisting of cetyltrimethylammonium bromide (CTAB) and hexadecyltrimethylammonium chloride (CTAC).

The solvent may be one or more selected from the group consisting of ethanol and distilled water.

The reactant may be washed more than twice using water and ethanol.

The catalyst may be one or more selected from the group consisting of sodium chloride, ammonia solution, and triethanolamine.

The preparing of the porous silica nanoparticles may include reacting the reactant in a mixture including hydrochloric acid and ethanol for 10 to 24 hours. When reacting with the mixture of hydrochloric acid and ethanol as described above, the surfactant may be removed to form a porous structure.

In the preparing of the porous silica nanoparticles, porous silica nanoparticles having a diameter of 50 to 300 nm or 50 to 200 nm may be prepared.

The binding of the amphipathic polymer may include mixing the silica nanoparticles and the amphipathic polymers in a volume ratio of 100:20 to 80 or a volume ratio of 100:40 to 60 to bind the amphipathic polymer to the porous nanoparticles.

The amphipathic polymer may include any one or more selected from the group consisting of silane-poly(ethylene glycol)-COOH, polyethyleneimine (PEI), chitosan, polypropylene imine, polyly sine, polyamidoamine, poly(allylamine), poly(di allyldimethyl ammonium chloride), poly(N-isopropyl acrylamide-co-acrylamide), poly(N-isopropylacrylamide-co-acrylic acid), diethylaminoethyl-dextran, poly(N-ethyl-vinylpyridinium bromide), poly(dimethylamino)ethyl methacrylate, poly(ethylene glycol)-co-poly(trimethylamino ethyl methacrylate chloride), and methoxy poly(ethylene glycol)-b-poly(D, L-lactide).

The binding of the amphipathic polymer may include mixing the silica nanoparticles and the amphipathic polymers and reacting the mixture at 30 to 80° C. for 20 to 30 hours, and performing washing additionally twice or more with water and ethanol.

The binding of the antibody may include reacting the amphipathic polymer-bound silica nanoparticles with a crosslinker and an antibody to bind the antibody to the amphipathic polymer.

The virus antibody may include antibodies of any one or more selected from the group consisting of influenza A virus, influenza B virus, dengue virus, human respiratory syncytial virus, norovirus, MERS coronavirus, SARS coronavirus, and SARS coronavirus-2.

The influenza A virus antibody may include any one or more selected from the group consisting of H1N1 virus antibody, H2N2 virus antibody, H3N2 virus antibody, H5N1 virus antibody, H7N9 virus antibody, H7N7 virus antibody, H1N2 virus antibody, H9N2 virus antibody, H7N2 virus antibody, H7N3 virus antibody, H5N2 virus antibody, and H10N7 virus antibody.

In the binding of the antibody, the reaction may be performed at 1 to 10° C.

The binding of the antibody may include bounding a carboxyl group of the amphipathic polymer and the antibody.

Hereinafter, the present disclosure will be described in more detail through example embodiments. These example embodiments are only for the purpose of describing the present disclosure in more detail, and it will be apparent to those skilled in the art that the scope of the present disclosure is not limited by these example embodiments according to the gist of the present disclosure.

### Example 1

1) Preparation of Silica Nanoparticles (SiNPs)

A modified Stöber method was applied to synthesize SiNPs. To synthesize SiNPs, ammonia solution, distilled water, and ethanol were mixed, and then a solution in which tetraethyl silicate mineral (TEOS) is dispersed in ethanol was quickly added and reacted at room temperature. After 5 hours, washing was performed 3 times using distilled water and ethanol. Dispersion was performed in distilled water for later use.

2) Preparation of Mesoporous Silica Nanoparticles (MSNs)

A modified sol-gel method was applied to synthesize MSNs. To synthesize MSNs, cetyltrimethylammonium bromide (CTAB), ammonium chloride, and ethanol were dispersed in distilled water and heated to 70° C. Afterwards, tetraethyl orthosilicate (TEOS) was slowly added and reacted. After 2 hours, washing was performed 3 times with water and ethanol. MSNs were added to the mixture of hydrochloric acid and ethanol to remove CTAB, and a reaction was performed overnight at 78° C. Thereafter, isolation was performed via centrifugation, followed by dispersion in ethanol.

3) PEGylation of Nanoparticles

SiNPs and MSNs were dispersed in a solution mixed with water and ethanol, respectively, to PEGylate SiNPs and MSNs. Subsequently, silane-poly(ethylene glycol)-COOH (silane-PEG-COOH) was added to each solution respectively and reacted at 60° C. After 24 hours, washing was performed 3 times with water and ethanol. In this case, SiNPs and polymers (silane-PEG-COOH) were mixed in a volume ratio of 50:28.

4) H1N1-Specific Antibody Binding

Binding of H1N1-specific antibodies to SiNP-PEG-COOH and MSN-PEG-COOH was performed by the EDC/NHS method. SiNP-PEG-COOH, MSN-PEG-COOH, EDC, sulfo-NHS, and antibodies were dispersed in DW and reacted at 4° C. After 6 hours, washing was performed with distilled water, and then dispersion was followed in a basic solution (distilled water and PBS) to be stored at 4° C. for later use.

### Experimental Example 1

In order to determine the form of silica-based nanostructures according to the present disclosure, dynamic light scattering (DLS) analysis and transmission electron microscope (TransTEM) imaging were performed for silica nanoparticles (SiNPs) and mesoporous silica nanoparticles (MSNs), PEGylated silica nanoparticles (SiNP-PEG), PEGylated mesoporous silica nanoparticles (MSN-PEG), antibody-PEGylated silica nanoparticles (SiNP-PEG-Ab), and antibody-PEGylated mesoporous silica nanoparticles (MSN-PEG-Ab) prepared in Example 1, the results of which are shown in FIGS. 2 to 4.

FIG. 2 shows results of identifying synthesized silica nanoparticles (SiNPs) and mesoporous silica nanoparticles (MSNs) through dynamic light scattering (DLS) and transmission electron microscope (TEM). In FIG. 2, it was found that silica-based nanoparticles with high dispersion and uniform size were formed through dynamic light scattering (DLS). In addition, in the observation of the morphology and surface of the synthesized nanoparticles through transmission electron microscope (TEM), the overall size was uniform and spherical, and in the case of mesoporous silica nanoparticles (MSNs), the pore structure appeared.

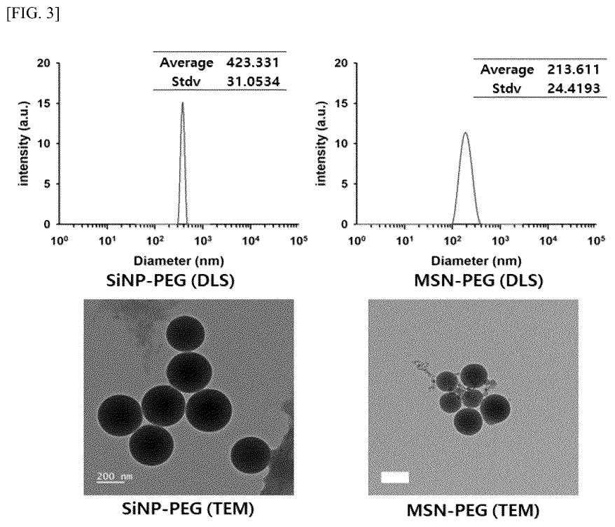

FIG. 3 shows results of identifying SiNP-PEG-COOH and MSN-PEG-COOH synthesized after carrying out PEGylation through dynamic light scattering (DLS) and transmission electron microscope (TEM). In FIG. 3, it was found that PEGylation was achieved through an increase in the particle size of the dynamic light scattering (DLS). Through transmission electron microscopic images, it was found that the pore structure which is the most important in MSN-PEG-COOH was maintained.

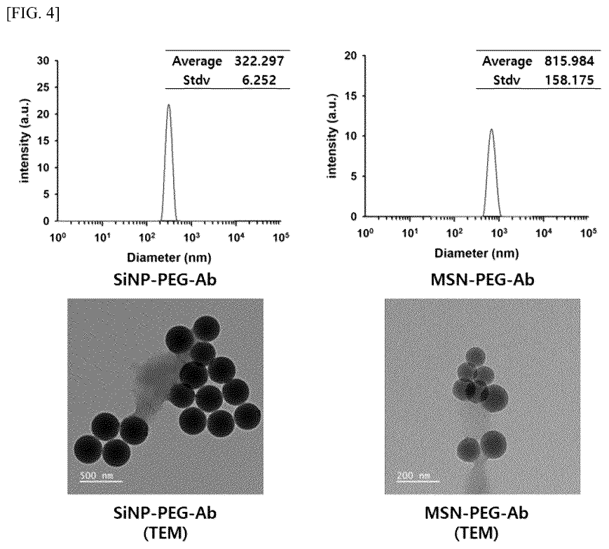

FIG. 4 shows results of identifying the antibody-PEGylated silica nanoparticles (SiNP-PEG-Ab) and antibody-PEGylated mesoporous silica nanoparticles (MSN-PEG-Ab) generated after antibody binding through dynamic light scattering (DLS) and transmission electron microscope (TEM). Through transmission electron microscopic images, it was found that the pore structure which is the most important in MSN-PEG-Ab was maintained.

### Experimental Example 2

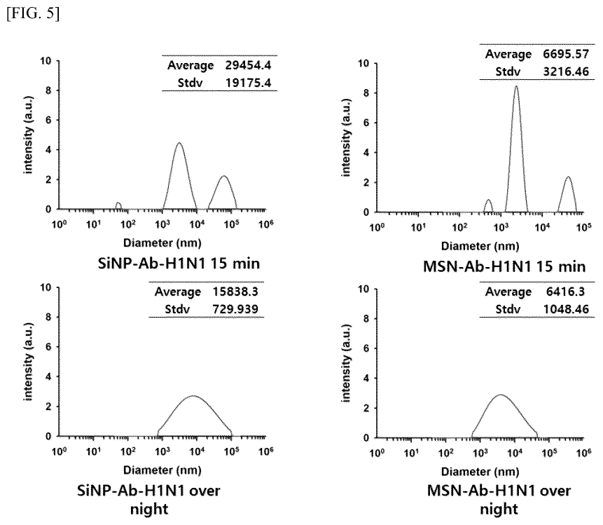

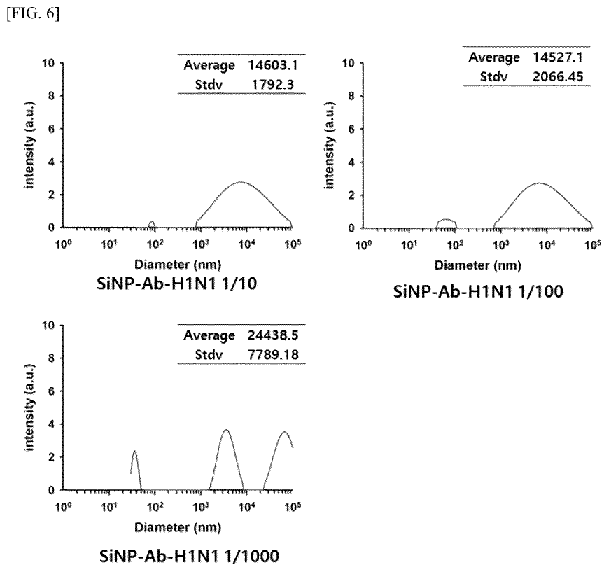

In order to identify the binding and agglomeration between silica-based nanostructures according to the present disclosure and H1N1 viruses, antibody-PEGylated silica nanoparticles (SiNP-PEG-Ab) and antibody-PEGylated mesoporous silica nanoparticles (MSN-PEG-Ab) prepared in Example 1 were reacted with the H1N1 virus respectively, and dynamic light scattering (DLS) analysis was performed to detect the agglomeration, the results of which are shown in FIGS. 5 to 7. Specifically, the experiment with each concentration of viruses was carried out by diluting the virus concentration by 10, 100, and 1000 times.

In FIG. 5, several peaks appeared 15 minutes after the reaction with the virus, but it was found that the particle size increased overall. Further, when the viral reaction was carried out overnight, it was found that it appeared in a single peak, and the size of the dynamic light scattering (DLS) image also enlarged.

In FIG. 6, antibody-PEGylated silica nanoparticles were bound and agglomerated with H1N1 viruses at various concentrations and then analyzed for light scattering, and it was found that particle agglomeration normally occurred even in virus samples diluted 10, 100, and 1000 times.

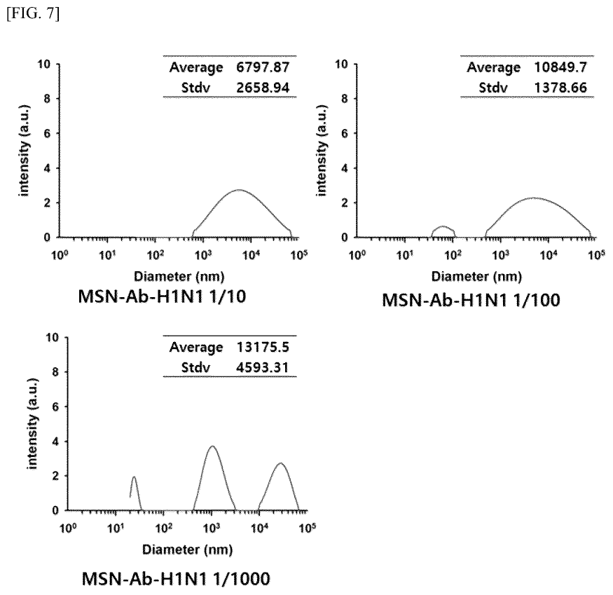

FIG. 7 shows results of performing light scattering analysis after antibody-PEGylated mesoporous silica nanoparticles were bound and agglomerated with various concentration of H1N1 viruses, and it was found that particle agglomeration normally occurred even in virus samples diluted 10, 100, and 1000 times.

Having described a specific part of the present disclosure in detail above, it is clear that for persons skilled in the art, such a specific description is only a preferred example embodiment and is not limited to the scope of the present disclosure. Thus, the substantive scope of the present disclosure will be defined by the attached claims and their equivalents.

The scope of the present disclosure is indicated by the appended claims, and the meaning and scope of the claims and all alterations or modified forms derived from the equivalent concept thereof should be construed as being included in the scope of the present disclosure.

1. A nanostructure for detecting viruses, comprising:
porous silica nanoparticles;
an amphipathic polymer bound to a surface of the silica nanoparticles; and
a virus antibody bound to the amphipathic polymer.

2. The nanostructure of claim 1, wherein the amphipathic polymer comprises any one or more selected from the group consisting of silane-poly(ethylene glycol)-COOH, polyethyleneimine (PEI), chitosan, polypropylene imine, polylysine, polyamidoamine, poly(allylamine), poly(diallyldimethylammonium chloride), poly(N-isopropyl acrylamide-co-acrylamide), poly(N-isopropylacrylamide-co-acrylic acid), diethylaminoethyl-dextran, poly(N-ethyl-vinylpyridinium bromide), poly(dimethylamino)ethyl methacrylate, poly(ethylene glycol)-co-poly(trimethylamino ethyl methacrylate chloride), and methoxy poly(ethylene glycol)-b-poly(D, L-lactide).

3. The nanostructure of claim 1, wherein the virus antibody comprises antibodies of any one or more selected from the group consisting of influenza A virus, influenza B virus, dengue virus, human respiratory syncytial virus, norovirus, MERS coronavirus, SARS coronavirus, and SARS coronavirus-2.

4. The nanostructure of claim 3, wherein the influenza A virus antibody comprises any one or more selected from the group consisting of H1N1 virus antibody, H2N2 virus antibody, H3N2 virus antibody, H5N1 virus antibody, H7N9 virus antibody, H7N7 virus antibody, H1N2 virus antibody, H9N2 virus antibody, H7N2 virus antibody, H7N3 virus antibody, H5N2 virus antibody, and H10N7 virus antibody.

5. The nanostructure of claim 1, wherein a carboxyl group of the amphipathic polymer and the antibody are bound and linked.

6. The nanostructure of claim 1, wherein the porous silica nanoparticles have a diameter of 50 to 300 nm.

7. A method of diagnosing viral infection, comprising:
reacting a composition comprising the nanostructure, as an active ingredient, prepared in claim 1 to a virus; and
detecting agglomeration of the virus.

8. A method of preparing a nanostructure for detecting viruses, comprising:
preparing a porous silica nanoparticle by adding a silica precursor to a mixture of surfactants, solvents, and catalysts to carry out a reaction and then washing the reactants;
mixing the porous silica nanoparticle with an amphipathic polymer so that the amphipathic polymer is bound to a surface of the porous silica nanoparticle; and
mixing the amphipathic polymer-bound silica nanoparticle with a virus antibody so that virus antibody is bound to the amphipathic polymer.
